SEQUENCE LISTING (3 sequences)

SEQ ID NO: 1 - DNA, 3569 bp, Arabidopsis thaliana
gatctttcagagaaaaaaaataaattttttttgacaaacgtagtgctaaactaaaccgtaaaaaaaaaggaaaaaaatgtcccttattcagatttccttttgtaacccacacacatagctaaactaatttacatcataattaaccactaaccagtgtcacgaccttgcttcattggtcttaaaaaggtccatgtagggtcgtcagaaaagtaaaaaagaattataatgcaataggattaattatccaattagctgattaagtctaaatcaagctgtctaagtggtgacgaaaacaaaacaagcttattcaacactagatttgttaattggattattgaaattgtaatgaaatgacgagtggttgatgaataaagggaaattaatgttatttaataaataaataaataaaatcatcacaggcgtatcggatctgtgactaaaatcaattattggctctgttatactgttactaatcaatatcaaagaatagatttatgccttcttgccattctcagtgccactgaaaaagttttttcctatctaatttatttttgttccaaatattaattcaaaccataaaatatgtatatgctacatatgcagtgagacatttaatgatcgaaggagccattgattgaacacaattaggaacaccatgcatcttatctacaatttccaatatcttcttgtaatactcaaagtcaaaagattggatctacaatctgacgaaagaaataaagaaacgcttacacagtctttttttcatttcaccaacatgtattattatctcacatttgaatctaaatagtaacacaacaatatcagcacaaaccaattacatatttttcgtattataatatatttttttcatatcgattacaatcttaacgtcgttttataaaataaatttggggtttttttttgttaaagggttttaaaacaaaatttgttccaagttaaatgtcgttcaaaaatttaatggaatatatatatatatatatatatatttttagaaaacactagttatagaattaaaatggataaaaatatgttattttaattgaacatatatacatcgaaactttttgttggttttgttagcgtttagcgatgttgagctacgagttctattgatggttgtttacaacaataattggattggagaacaagaagttatacatgattcgtgaagttaattagataagtttttaatacgaagaaatgagtcccgagacaaaaatgaagcttatggaattaattggtaaattagcatggcgacatacatttgtgttatgaaatcatctagttgtaggcacggtgatggatccctcagatggtcatgctatcattttcgctttcaaatagcgcgacctaattttttatataataaaattactaacgtggatcgcatgggatattttaatataataaaaatgttttaagaaaataaggaaatggaagagcccaccgtccaccaataaattaccgagtaaacgatttatacgaccgtcgaaatgaactgagaagataacgagaaaaaaagaatcggaattatatattttgactcaaaaacgagaaaataattcgtagcgattctaactcctactttataccttaaggaacacgaaacttatgagattttatggaagttacaacgtggttagtttttttttctttctattggaccagtgttaaattttcaatttggcatggtgtaaaactacacaaaacagcctttctttctctgacccgtaaaactactattttatcttatttcaaatctaacagattttcattatggcgatagatatagtccttaaaaattatattggattcattagcaaaacataactatacattgaaattgtattgataaaatttatattattacatgcaaccaagcaagagcggatgtacacgttttggtgtgggtgcgagttccacatcagaatttgtttgtctatataagtaattgtgagagacaatcggaataattggctagaatcagtctttttttcctagtggatctttaaaaaccattcttttataccaagcatgtacatgctgtggtgtgggtgtaagtaaatcctgccccaatgaaaattgtttttggactcgccactgcaacgaagtgtaccaacaacttgactaggattctaagttcttttatgtataggatgtctatattaaactaccatgactaacatatatatagtagttccatatgctcgataaactatgatagatcaacaattttaaacatatagtttaacactatttatttgttcaacgtcaatagtttatagttcgcatgcgctcggcttagatttggtccccaacagtcgaaattgtcaaataatataaaataaaagtttcattgttaggattcatttattcttcgggtggttattgtaataaaaggcaaaagaaaaagaagaacaaaattcacaagtaaaaaaaaagataacatcattcttttagtcgacaaaaaaaaaaaaaaaatcaaaaagatttattcagtactacagtttaatattgttttgacttttttctttttctttatattatctgaaaattctagactgcagctgaaacatgtgatatggattaaaggcgtatccagtatccacagaaagaggagtggtgtcgctcacccagtcacccttgttacttgttagatagcattaatacatttgtaagcaacagcttatctaatagacatgtcttaattgggaaatatgctctaagatgatacaaccatggttccaactgttgaccaccataactgataacatgttgattacattttttcttttcagttacaacgattacttttttggggaaattattgatataatatgattcattggatgatccgatatcatgcatataaagttgtatctcgtgaaacacgagatagtattatactccattctttcattatcggagtatgtttaaaatttgaaaacaaatacagacacggaccgtggtctttaccttcagaaaaaaaaagagaaaaaaaaacaatccactgtttattataggagttgtagaaaatcgggcaacgatattcgatatgagttattattagggccttattattatatggtattactggatattactaaataatcatataaatatcacattttaatatacactcgttggacacgcggaatattatatgttctaaatgttaaaaaatcaacagaatacaacgatcgacggatctagagtctagaccatgcaaatacctcatcctatttacatataataactgtgcatatagtttagtcaaataaaaaggtaaagaaacaatatacaacctataacgtcaatatccatgtacgtagtaataattaggatatgacacgaacacacgatatcttgatatatacaaaatgaaaacttaaaaattgattaatatggcctggctgggtatattattaaaaaaacataaagagagatcaataattgattcgaagatcactatataaagaacgtcttcgatatgtaaaagaaccatcctaaacattttttcttgaataaaatcagaattacaaacaaaa

SEQ ID NO: 2 - DNA, 1448 bp, Arabidopsis thaliana
ctgcaacgaagtgtaccaacaacttgactaggattctaagttcttttatgtataggatgtctatattaaactaccatgactaacatatatatagtagttccatatgctcgataaactatgatagatcaacaattttaaacatatagtttaacactatttatttgttcaacgtcaatagtttatagttcgcatgcgctcggcttagatttggtccccaacagtcgaaattgtcaaataatataaaataaaagtttcattgttaggattcatttattcttcgggtggttattgtaataaaaggcaaaagaaaaagaagaacaaaattcacaagtaaaaaaaaagataacatcattcttttagtcgacaaaaaaaaaaaaaaaatcaaaaagatttattcagtactacagtttaatattgttttgacttttttctttttctttatattatctgaaaattctagactgcagctgaaacatgtgatatggattaaaggcgtatccagtatccacagaaagaggagtggtgtcgctcacccagtcacccttgttacttgttagatagcattaatacatttgtaagcaacagcttatctaatagacatgtcttaattgggaaatatgctctaagatgatacaaccatggttccaactgttgaccaccataactgataacatgttgattacattttttcttttcagttacaacgattacttttttggggaaattattgatataatatgattcattggatgatccgatatcatgcatataaagttgtatctcgtgaaacacgagatagtattatactccattctttcattatcggagtatgtttaaaatttgaaaacaaatacagacacggaccgtggtctttaccttcagaaaaaaaaagagaaaaaaaaacaatccactgtttattataggagttgtagaaaatcgggcaacgatattcgatatgagttattattagggccttattattatatggtattactggatattactaaataatcatataaatatcacattttaatatacactcgttggacacgcggaatattatatgttctaaatgttaaaaaatcaacagaatacaacgatcgacggatctagagtctagaccatgcaaatacctcatcctatttacatataataactgtgcatatagtttagtcaaataaaaaggtaaagaaacaatatacaacctataacgtcaatatccatgtacgtagtaataattaggatatgacacgaacacacgatatcttgatatatacaaaatgaaaacttaaaaattgattaatatggcctggctgggtatattattaaaaaaacataaagagagatcaataattgattcgaagatcactatataaagaacgtcttcgatatgtaaaagaaccatcctaaacattttttcttgaataaaatcagaattacaaacaaaa

SEQ ID NO: 3 - DNA, 788 bp, Arabidopsis thaliana
ataactgataacatgttgattacattttttcttttcagttacaacgattacttttttggggaaattattgatataatatgattcattggatgatccgatatcatgcatataaagttgtatctcgtgaaacacgagatagtattatactccattctttcattatcggagtatgtttaaaatttgaaaacaaatacagacacggaccgtggtctttaccttcagaaaaaaaaagagaaaaaaaaacaatccactgtttattataggagttgtagaaaatcgggcaacgatattcgatatgagttattattagggccttattattatatggtattactggatattactaaataatcatataaatatcacattttaatatacactcgttggacacgcggaatattatatgttctaaatgttaaaaaatcaacagaatacaacgatcgacggatctagagtctagaccatgcaaatacctcatcctatttacatataataactgtgcatatagtttagtcaaataaaaaggtaaagaaacaatatacaacctataacgtcaatatccatgtacgtagtaataattaggatatgacacgaacacacgatatcttgatatatacaaaatgaaaacttaaaaattgattaatatggcctggctgggtatattattaaaaaaacataaagagagatcaataattgattcgaagatcactatataaagaacgtcttcgatatgtaaaagaaccatcctaaacattttttcttgaataaaatcagaattacaaacaaaa

(19) United States
(12) Patent Application Publication
(10) Pub. No.: US 2003/0177536 A1
(43) Pub. Date: Sep. 18, 2003

(54) Root-specific promoter

(76) Inventors: Florian Grundler, Monkeberg (DE); Inke Nitz, Kiel (DE); Piotr Puzio, Berlin (DE)

Correspondence Address: FULBRIGHT & JAWORSKI L.L.P., 600 CONGRESS AVE., SUITE 2400, AUSTIN, TX, 78701, US

(21) Appl. No.: 10/168,349

(22) PCT Filed: Dec. 18, 2000
(86) PCT No.: PCT/DE00/04521

(30) Foreign Application Priority Data
Dec. 16, 1999 (DE) 199 60 843.1

(51) Int. Cl. A01H 1/00; C12N 15/82; C07H 21/04

(52) U.S. Cl. 800/288; 435/320.1; 435/468; 536/23.2

(57) ABSTRACT

The present invention relates to transgenic plants exhibiting a regulatory nucleic acid sequence according to SEQ ID NOs: 1 to 3 stably integrated into its genome after its transformation, or to a fragment or derivative thereof, and to a nucleic acid sequence encoding a gene product, said nucleic acid sequence being operably linked to said regulatory nucleic acid sequence. Furthermore, the present invention relates to methods for the preparation of transgenic plants according to the present invention and to nucleic acid sequences according to SEQ ID NOs: 1 to 3. The regulatory nucleic acid sequence relates to polynucleotides naturally enabling in plants of the species Arabidopsis thaliana largely root-specific expression of a foreign gene.

The present invention relates to transgenic plants exhibiting a regulatory nucleic acid sequence according to SEQ ID NOs: 1 to 3 stably integrated into its genome after its transformation, or to a fragment or derivative thereof, and to a nucleic acid sequence encoding a gene product, said nucleic acid sequence being operably linked to said regulatory nucleic acid sequence. Furthermore, the present invention relates to methods for the preparation of transgenic plants according to the present invention and to nucleic acid sequences according to SEQ ID NOs: 1 to 3. The regulatory nucleic acid sequence relates to polynucleotides naturally enabling in plants of the species Arabidopsis thaliana largely root-specific expression of a foreign gene.

Promoters function, amongst others in plants, as regulators of the transcription of natural and recombinant genes. The entirety of all DNA segments regulating the specificity of the transcription of a gene is termed the promoter of the gene, wherein distinct regulatory elements are distinguishable within the promoters. The promoters govern the spatial and temporal transcription of the genes, that is at which location of the plant and when during its development the genes regulated by them are expressed.

It is known from public prior use to introduce foreign genes into plants in order to express them. Often, constitutive promoters are employed in such transgenic plants, which promoters entail a permanent expression of the gene in almost all tissues of the plant. It may be desirable for various reasons such as the amelioration of the gene expression and the increase of the concentration of a protein expressed from a foreign gene, such as the amelioration of the energy balance (protein is expressed only at locations where it is needed) and the amelioration of the environmental security, to express foreign genes not constitutively but in a tissue-specific manner. For example, it may be desirable to protect roots from pathogens or parasites by means of particular polypeptides, or to enrich specific polypeptides for their isolation in roots.

The present invention is thus based on the object to provide polynucleotides exhibiting a nucleic acid sequence enabling a root-specific expression of transgenes in plants. The object is solved by the subject-matter as defined in the claims.

The invention is explained by means of the following figures.

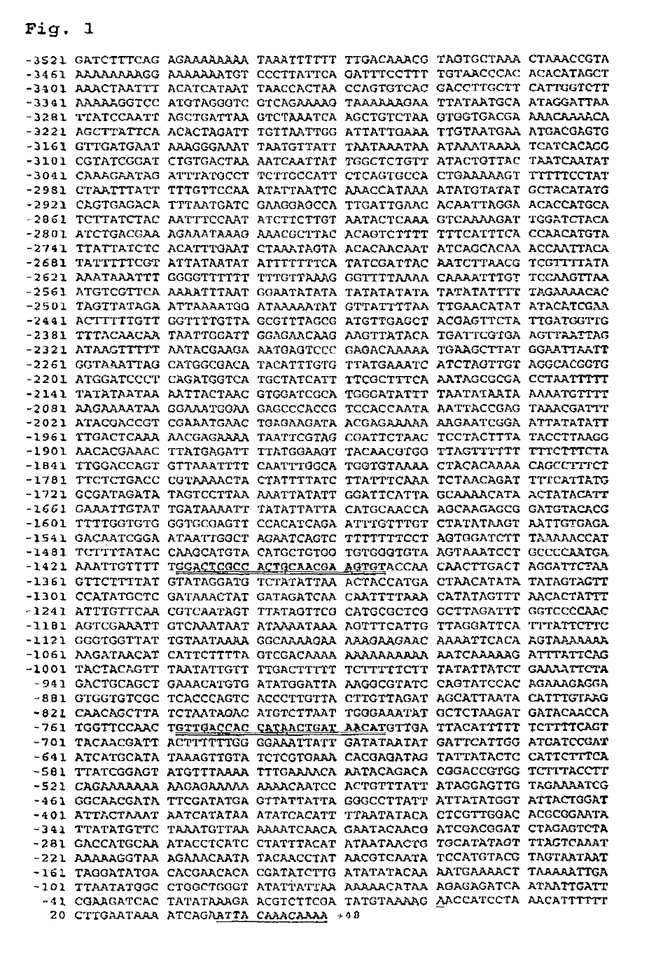

FIG. 1 depicts the nucleic acid sequence of the region located in 5′ position upstream the transcription initiation site of the PyK10 gene. In the following, this region will also be termed pPYK10 and is depicted in SEQ ID NO: 1. Position 1 identifies the transcriptional start site. The underlined base in italics identifies position-1 in front of the transcriptional start site. The doubly underlined region in bold type letters identifies primer sequences modified for their insertion into an XhoI cleavage site. The underlined region in bold type letters identifies the reverse primer sequence.

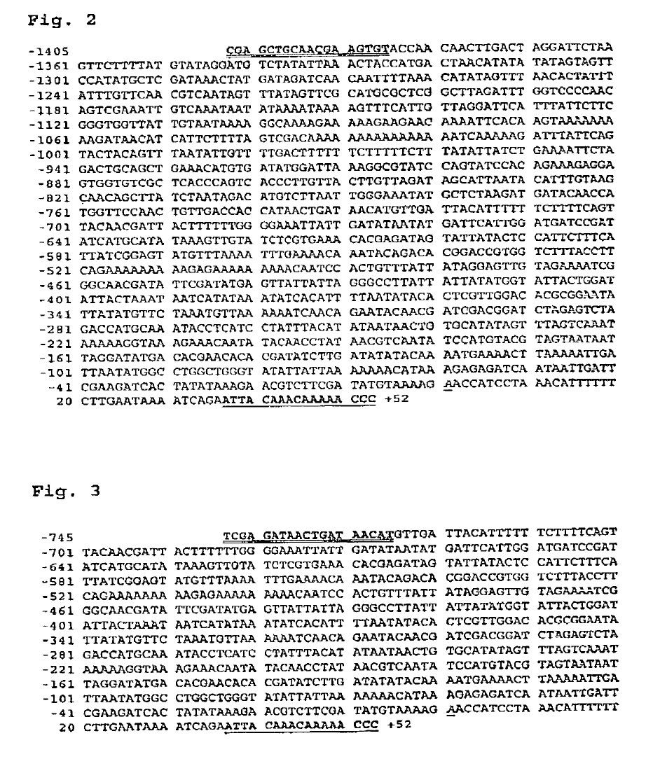

FIG. 2 identifies the nucleic acid sequence of a fragment of pPYK10. In the following, this fragment will also be termed pPYK10c and is depicted in SEQ ID NO: 2 without the restriction enzyme cleavage sites introduced. Position 1 identifies the transcriptional start site. The underlined base in italics identifies position-1 in front of the transcriptional start site. The doubly underlined region in bold type letters identifies a primer sequence modified for its insertion into an XhoI cleavage site. The underlined region in bold type letters identifies the reverse primer sequence.

FIG. 3 identifies the nucleic acid sequence of a fragment of pPYK10 and pPYK10c. In the following, this fragment will also be termed pPYK10b and is depicted in SEQ ID NO: 3 without the restriction enzyme cleavage sites introduced. Position 1 identifies the transcriptional start site. The underlined base in italics identifies position-1 in front of the transcriptional start site. The doubly underlined region in bold type letters identifies a primer sequence modified for its insertion into an XhoI cleavage site. The underlined region in bold type letters identifies the reverse primer sequence.

The term “operably linked” as used herein means that a regulatory sequence such as a promoter regulates the expression of a gene. The term “transgenic plant” as used herein means plants produced by means of recombinant DNA technology and/or microbiological methods but not by means of conventional methods of breeding.

The term “vector” as used herein means naturally occurring or artificially produced constructs for the uptake, proliferation, expression, or transfer of nucleic acids, e.g., plasmids, phagemids, cosmids, artificial chromosomes, bacteriophages, viruses, and retroviruses.

The terms “homologues” and “homologous sequences” as used herein mean nucleic acid sequences with a significant similarity to a reference sequence or portions thereof. Accordingly, nucleic acid sequences hybridizing to the reference sequences or portions thereof under stringent or less stringent conditions (for a definition of stringent and less stringent conditions reference is made to Sambrook et al., Molecular Cloning, Cold Spring Harbor Laboratory (1989), ISBN 0-87969-309-6) are homologous sequences. An example for stringent hybridization conditions is as follows: hybridization in 4×SSC at 65° C. (in the alternative, in 50% formamide and 4×SSC and 42° C.), subsequently several washing steps in 0.1×SSC at 65° C. over a period of altogether about one hour. An example for less stringent hybridization conditions is a hybridization in 4×SSC at 37° C. and subsequently several washing steps in 1×SSC at room temperature. Furthermore, homologous sequences are nucleic acid sequences or portions thereof exhibiting a significant similarity to a reference sequence when applying the similarity algorithm BLAST (Basic Local Alignment Search Tool, Altschul et al., Journal of Molecular Biology 215, 403-410 (1990)). Significantly similar, as used herein, are sequences exhibiting, e.g., by means of standard parameters in the BLAST service of the NCBI, an identity of at least 60%, as compared to the reference sequence. In other words, the sequences exhibit a degree of homology of at least 60%.

The polynucleotides according to the present invention are functionally defined by the feature that they allow in plants of the species Arabidopsis thaliana a largely root-specific expression of a foreign gene (hereinafter also termed transgenes). “Largely” within the meaning of the present invention means that the expression of the transgene in the roots significantly preponderate an expression conceivable in shoot organs of the plant. The expression in the root significantly preponderates within the meaning of the present invention, if it is at least twice as strong as it is in the shoot organs. The promoter activity of the polynucleotides according to the present invention may be limited to distinct root tissues or root regions such as the roots tips, the lateral root buds and the like. The promoter activity may, however, also extend to the entire plant root without limitation to particular regions or tissues. It is conceivable within the scope of the present invention that a polynucleotide according to the present invention exhibits an unspecific phase, for example, at the outset of the development of a transgenic plant, that is a promoter activity not limited to the root. In this context, it should be noted that the above functional feature does not include a limitation of the product claim, directed to a polynucleotide, to a use only in plants of the species Arabidopsis thaliana. Rather, the polynucleotides according to the present invention can be used for the preparation of transgenic dicot plants, in particular plants of the family Brassicaceae, e.g., of the genera Brassica, Sinapis, or Raphanus, or of the family Solanaceae, e.g., of the genera Lycopersicon, Solanum, or Capsicum, or of the family Fabaceae, e.g., of the genera Vicia, Medicago, Trifolium, Glycine, or Pisum, or of the family Cucurbitaceae, e.g., of the genera Cucumis or Cucurbita, or of the family Apiaceae, e.g., of the genera Daucus or Apium, or of the family Rosaceae, e.g., of the genera Malus, Pyrus, Rubus, Fragaria, or Prunus, or of the family Convulvulaceae, e.g., of the genus lpomoea, or of the family Euphorbiaceae, e.g., of the genus Manihot, or of the family Chenopodiaceae, e.g., of the genus Beta.

Additionally, the polynucleotide according to the present invention can be used for the preparation of transgenic monocot plants, in particular plants of the family Poaceae, e.g., of the genera Triticum, Hordeum, Avena, Secale, Oryza, Zea, or Saccharum, or of the family Musaceae, e.g., of the genus Musa, or of the family Arecaceae, e.g., of the genera Phoenix, Elacis, or Cocos.

The polynucleotides according to the present invention are additionally characterized by any one of the following four features:

a) at least 200 contiguous nucleotides as derivable from SEQ ID NO: 1,

b) at least 200 nucleotides homologous with a corresponding contiguous sequence as derivable from SEQ ID NO: 1 to a degree of at least 60%,

c) polynucleotides, obtainable by screening a DNA library of a plant of the family Brassicaceae with a gene probe exhibiting at least 50 nucleotides of a contiguous nucleic acid sequence derivable from SEQ ID NO: 1,

d) promoter activity exhibiting fragments of the polynucleotides defined in c). The minimum length of a polynucleotide according to the present invention according to features a) or b) is 200 nucleotides. Preferred minimum lengths are 300, 400, 500, 600, and 700 nucleotides, respectively.

According to feature b), a polynucleotide according to the present invention exhibits a homology of at least 60% to a corresponding number of nucleotides as derivable from SEQ ID NO: 1. Further preferred is a homology of at least 70%, 80%, and 90%, respectively. A criterium applied optionally independent on the degree of homology, is whether a polynucleotide as a single strand can hybridize under stringent conditions with a single strand of a corresponding length as derivable from SEQ ID NO: 1.

According to feature c), a subject-matter of the present invention is further a polynucleotide obtainable by screening of a DNA library of a plant of the family Brassicaceae with a corresponding gene probe. As an example, such DNA libraries may be of the genus Arabidopsis, within this genus DNA libraries of the species Arabidopsis thaliana. Such DNA libraries are readily available to the skilled person. Subject-matter of the invention are further fragments of the polynucleotides obtained by means of the above gene probe, provided they exhibit a largely root-specific promoter activity. A preferred length of such promoter activity exhibiting fragments is likewise 200 nucleotides.

According to the present invention, a polynucleotide having the biological function of a promoter is provided, the polynucleotide expressing in transgenic plants an operably linked foreign gene in a largely root-specific manner. Thus, one may enrich distinct polypeptides specifically in roots, or one may influence by means of such polypeptides for example the growth of the roots or increase their resistance or defense to pathogens and parasites.

“Foreign gene” according to the present invention means that nucleic acid sequences encoding a gene product both endogenously and exogenously may be used. Endogenous means that the nucleic acid sequence originates from the same organism into which they are integrated according to the present invention. Exogenous means, however, that the nucleic acid sequence originates from another organism.

The polypeptides prepared and/or enriched specifically in the roots and optionally isolated may originate from any organism such as humans, animals, plants, fungi, protozoae, or viruses and may be any polypeptide.

Polypeptides capable to influence the growth of the roots may be, e.g., growth factors, plant hormones, inhibitors, or enzymes of the secondary metabolism.

Pathogens or parasites that are to be debilitated or defended by the polypeptides specifically expressed in the roots may be soil born fungi, e.g., of the genus Pythium, Fusarium, or Verticillium, or soil born protozoae, e.g., of the genera Plasmodiophora or Spongospora, or plant parasitic nematodae, e.g., of the genera Globodera, Heterodera, Pratylenchus, Radopholus, Trichodorus, or Longidorus, or insects, e.g., of the genera Melolontha, Otiorhynchus, or Tipula.

Upon introduction of such a promoter into the plants to be modified, generally only the expression of the fused foreign gene is regulated. No pleiotropic promoter effects are to be expected. The quality of the breeding material of the cultured plant at issue is thus not affected as long as it is not influenced by the desired root-specific expression of the foreign gene.

Preferred polynucleotides useful according to the present invention are depicted in SEQ ID NO: 1, SEQ ID NO: 2, and SEQ ID NO: 3. Additionally, polypeptides exhibiting to these sequences a degree of homology of at least 60%, preferably 70%, still more preferred 80%, and even more preferred 90%, are also preferred.

Another aspect of the present invention is a vector containing not only a foreign gene to be introduced into a plant cell or plant tissue but also a polynucleotide according to the present invention operably linked thereto. The invention further relates to plant cells containing such vector or the polynucleotide according to the present invention stably integrated into the genome and the operably linked foreign gene, as well as to transgenic plants containing such plant cells. Transgenic plants according to the invention can be dicot or monocot plants of the families and genera listed previously. According to a preferred embodiment of the invention the transgenic plant is of the genus Arabidopsis, a plant of the species Arabiclopsis thaliana being one example only.

Furthermore, another aspect of the present invention is the use of a polynucleotide according to the present invention for the root-specific expression of a foreign gene in a plant and a process to prepare such transgenic plant, the process comprising the following steps: fusing a foreign gene to a polynucleotide according to the present invention, optionally preparing a vector containing the fusion product, introducing the vector or the fusion product into a plant cell or plant tissue, and regenerating the plant cell or the tissue to a plant, in particular to a fertile plant.

The invention will be explained in more detail by reference to the following working examples.

### EXAMPLE 1

### Isolation and Cloning of Promoter pPYK10

A genomic library of Arabidopsis thaliana wild-type C24 was used to isolate the promoter pPYK10. A 750bp HindIII restriction fragment derived from the 5′-region of the Pyk10 cDNA clone was used as a probe in order to screen the library. The plaque hybridization brought about a positive clone on the parent plate. The clone designated lambda-g1c was purified and isolated by means of two to three re-plating steps. The phage DNA was subjected to a restriction enzyme analysis, and the five fragments harboring the promoter sequences were subcloned in the vector pBluescript-M13+.

The 3′ terminus of the promoter was identified by means of the primer extension analysis (adenine-1).

### EXAMPLE 2

### Identification of Cis-Regulatory Sequence Elements

The promoter sequence pPYK10 was analyzed for putative cis regulatory sequence elements. Cis regulatory sequence elements are largely relatively short sequences which positively or negatively influence gene expression by their interaction with specific DNA binding proteins, the trans factors. The sequence analysis evaluated, apart from the TATA and the CAAT box, various cis elements located upstream and exhibiting homologies to regulatory sequences known from other eukaryotic promoters. The specific sequence elements are compiled in the survey of Table 1. All of the cis sequences listed therein are transcriptional activator sequences, except the repressor elements GAAAGAA and ATGGG.

Tab. 1: Compilation of the relevant cis regulatory sequence elements in the 5′ region of the Pyk10 gene. The transcription factors were, if known, listed. DR=direct repeat.

| | | | |
|---|---|---|---|
| Sequence | Elements | Number | Trans factors |

| | | | |
|---|---|---|---|
| ACGT | ACGT-Cores | 9 | e.g. GBFs, HBPs, CPRFs |
| CATTTG | CANNTG-Motif | 3 | CAN |
| CAGATG | CANNTG-Motif | 1 | CAN |
| CATATG | CANNTG-Motif | 1 | CAN |
| CAGCTG | CANNTG-Motif | 1 | CAN |
| CATGTG | CANNTG-Motif | 1 | CAN |
| CAACTG | CANNTG-Motif | 1 | CAN |
| GATA | GATA-repeat | 1 × 3 DR, 3 × 2 DR | ASF-2, |
| TGACG | as-1 element | 3 | ASF-1, TGA1-Fam. |
| TTATTCA | AP-1 elements | 1 | AP-1 |
| AAGTCT | AP-1 elements | 1 | AP-1 |
| TGAATAA | AP-1 elements | 2 | AP-1 |
| TGATTCA | AP-1 elements | 1 | AP-1 |
| TAACTG | Myb motif | 2 | MYB proteins |
| TAACAG | Myb motif | 1 | MYB proteins |
| CCAAT | | 3 | C/EBP |
| TGTAAT | | 1 | C/EBP |
| TGTCAC | | 1 | |
| TATTTTG | | 2 | |
| ATCTAAT | elicitor boxL | 1 | |
| ATTGTTT | elicitor box | 2 | |
| TTGACC | elicitor box | 1 | WRKY-Fam. |
| CCGTCC | elicitor box | 1 | |
| CTCC | elicitor box | 1 | |
| GTGTC | | 2 | VP1 |
| AAACCA | ARE sequence | 2 | |
| TGGTTT | ARE sequence | 1 | |
| GAAAGAA | | 1 | |
| ATGGG | ATGGG-Core | 1 | |

The ACGT core motifs are sequences contained in many plant genes and mediating signals of environmentally and developmentally caused stimuli. The CANNTG motifs regulate the spatial gene expression, the temporal gene expression, and the gene expression induced by light. The C/EBP elements mediate a cell type specific gene activity. Myb motifs control the secondary metabolism, regulate the cellular morphogenesis, and are involved in the signal transduction pathways of plant growth regulators. Elicitor boxes mediate a gene induction caused by elicitors. The regulatory sequence TATTTTG is an element responsive to wounds. The CTCC element is responsive both to wounds and elicitors. Genes exhibiting as-1 elements in their promoter may be induced by auxin, salicylic acid, and methyl jasmonate. Promoters exhibiting the sequence elements GTGTC or TGTCAC may be induced by abscisic acid and auxin, respectively. AP-1 elements can be found in several eukaryotic promoters, their function is not described in any detail, however. Since they exist to some extent also in the promoter regions of the myrosinases TGG1 and TGG2, they were considered to be relevant sequence elements. Sequences repeatedly occurring as direct and/or indirect repeats often are cis regulatory elements. In the sequence under examination several direct repeats of the sequence element GATA occur. GATA motifs are present in many promoter regions of dicots and are described as light- and tissue-specific elements. In FIG. 2 the arrangement of the cis elements occurring in the 5′ region of the Pyk10 gene is schematically depicted. For the sake of an easy survey only the most important regulatory sequences have been indicated.

### EXAMPLE 3

### Analysis of the Promoter Activity by Means of a Gus Reporter Gene Fusion

A promoter assay employing the GUS reporter gene system (Jefferson, 1987) was used in order to analyze the promoter activity of the gene PYK10. A promoter fragment was prepared, fused with the GUS gene and introduced into Arabidopsis thaliana by means of Agrobacterium tumefaciens.

Using the transgenic plants the organ and tissue specific GUS gene activity was to be determined. The fragments pPYK10b and pPYK10c destined for the cloning step (see above) were prepared via PCR. Amplification of the fragments was done with the Pfu polymerase, which polymerase exhibits a proofreading activity. For the subsequent cloning of the fragment into the binary vector pMOG819 the primer used were provided with restriction sites, as illustrated in the following table.

### Primers Used for the Preparation of the 5′ Promoter Deletions

The respective restriction enzyme recognition site is emphasized by bold types. The downward arrow identifies the restriction enzyme recognition site.

| | | | |
|---|---|---|---|
| PromB | GTTGC↓TCGAGATAACTGATAACAT | for | XhoI |
| PromC | GGACC↓TCGAGCTGCAACGAAGTGT | for | XhoI |
| PromF | TGCACCC↓GGGTTTTTGTTTGTAAT | rev | SmaI |

GUS Expression of the Promoter Fragment B

The promoter fragment C brought about a strong expression of GUS in the roots. The promoter constructs B and C exhibit similar expression patterns, wherein promoter construct B exhibits a smaller blue staining in cotelydons.

The regenerated plants were analyzed for their GUS expression. The result was that initially a blue staining occurred in the entire germ in plants harboring the promoter pPYK10c until a few days after the process of germination. The more the plant is developed, the more was the blue staining restricted to the roots, wherein the margins of the cotyledons still exhibited isolated faint blue stainings. In fully developed plants of Arabidopsis a blue staining occurred only in the roots, and the entire root system exhibited a GUS expression. Clones exhibiting the promoter pPYK10b have a similar expression pattern, but the cotyledons in early developmental stages exhibit a smaller staining.

1. A polynucleotide enabling a largely root-specific expression of a foreign gene in plants of the species Arabidopsis thaliana, selected from:
a) at least 200 contiguous nucleotides as derivable from SEQ ID NO: 1,
b) at least 200 nucleotides homologous with a corresponding contiguous sequence as derivable from SEQ ID NO: 1 to a degree of at least 60%,
c) polynucleotides, obtainable by screening a DNA library of a plant of the family Brassicaceae with a gene probe exhibiting at least 50 nucleotides of a contiguous nucleic acid sequence as derivable from SEQ ID NO: 1,
d) promoter activity exhibiting fragments of the polynucleotides defined in c).

2. Polynucleotide according to claim 1, characterized in that it is selected from SEQ ID NO: 1, SEQ ID NO: 2, SEQ ID NO: 3, and from polynucleotides exhibiting a degree of homology of at least 60% to said sequences.

3. Vector comprising a polynucleotide according to claim 1 or 2.

4. Transgenic plant with at least one polynucleotide according to claim 1 or 2 stably integrated into the genome subsequent to its transformation with a nucleic acid sequence encoding a gene product, said nucleic acid sequence being operably linked to the polynucleotide, or with a vector according to claim 3 present in the plant cells.

5. A transgenic plant according to claim for, wherein the plant is selected from the family Brassicaceae, in particular from the genera Brassica, Sinapis or Raphanus, or from the family Solanaceae, in particular from the genera Lycopersicon, Solanum, or Capsicum, or from the family Fabaceae, in particular from the genera Vicia, Medicago, Trifolium, Glycine, or Pisum, or from the family Cucurbitaceae, in particular from the genera Cucumis or Cucurbita, or of the family Apiaceae, in particular from the genera Daucus or Apium, or from the family Rosaceae, in particular from the genera Malus, Pyrus, Rubus, Fragaria, or Prunus, or from the family Convulvulaceae, in particular from the genus Ipomoea, or from the family Euphorbiaceae, in particular from the genus Manihot, or from the family Chenopodiaceae, in particular from the genus Beta, or from the family Poaceae, in particular from the genera Triticum, Hordeum, Avena, Secale, Oryza, Zea, or Saccharum, or from the family Musaceae, in particular from the genus Musa, or from the family Arecaceae, in particular from the genera Phoenix, Elaeis, or Cocos, or from the genus Arabidopsis in particular Arabiclopsis thaliana.

6. Transformed plant cell or transformed plant tissue with a vector according to claim 3 or with a polynucleotide according to claim 1 or 2 stably integrated into the genome subsequent to its transformation, and with a nucleic acid sequence encoding a gene product, said nucleic acid sequence being operably linked to the polynucleotide.

7. Transformed plant cell or transformed plant tissue according to claim 6, which can be regenerated to a fertile plant.

8. Seed, obtainable from plants according to claim 4 or 5.

9. Use of a polynucleotide according to claim 1 or 2, or of a vector according to claim 3 for the root-specific expression of a foreign gene in a plant.

10. Use according to claim 9, characterized in that the plant is selected from the family Brassicaceae, in particular from the stuff her genera Brassica, Sinapis or Raphanus, or from the family Solanaceae, in particular from the genera Lycopersicon, Solanum, or Capsicum, or from the family Fabaceae, in particular from the genera Vicia, Medicago, Trifolium, Glycine, or Pisum, or from the family Cucurbitaceae, in particular from the genera Cucumis or Cucurbita, or of the family Apiaceae, in particular from the genera Daucus or Apium, or from the family Rosaceae, in particular from the genera Malus, Pyrus, Rubus, Fragaria, or Prunus, or from the family Convulvulaceae, in particular from the genus Ipomoea, or from the family Euphorbiaceae, in particular from the genus Manihot, or from the family Chenopodiaceae, in particular from the genus Beta, or from the family Poaceae, in particular from the genera Triticum, Hordeum, Avena, Secale, Oryza, Zea, or Saccharum, or from the family Musaceae, in particular from the genus Musa, or from the family Arecaceae, in particular from the genera Phoenix, Elaeis, or Cocos, or from the genus Arabiclopsis in particular Arabidopsis thaliana.

11. Method for the preparation of a transgenic plant, the method comprising the following steps:
a) fusing a foreign gene to a polynucleotide according to claim 1 or 2,
b) optionally preparing a vector containing the fusion product obtained in step a),
c) introducing the fusion product obtained in step a) or the vector obtained in step b) into a plant cell or plant tissue, and
d) regenerating the plant cell or the tissue to a plant, in particular to a fertile plant.
